(19) United States
(12) Patent Application Publication
(10) Pub. No.: US 2026/0053384 A1
(43) Pub. Date: Feb. 26, 2026

(54) DEVICE FOR NONINVASIVE IN VIVO MEASUREMENT OF PHYSIOLOGICAL VITAL SIGNS

(71) Applicant: Joseph Chaiken, Fayetteville, NY (US)

(72) Inventor: Joseph Chaiken, Fayetteville, NY (US)

(21) Appl. No.: 19/104,788

(22) PCT Filed: Aug. 18, 2023
(86) PCT No.: PCT/US2023/072456

§ 371 Date: Feb. 19, 2025

Related U.S. Application Data
(60) Provisional application No. 63/399,239, filed on Aug. 19, 2022.

(51) Int. Cl. A61B 5/0295; A61B 5/00; A61B 5/024; A61B 5/026

(52) U.S. Cl. CPC A61B 5/0295; A61B 5/0022; A61B 5/02416; A61B 5/0261; A61B 5/6826; A61B 5/742

(57) ABSTRACT

A device for noninvasive in vivo measurement of physiological vital signs that can provide the fluid content of the intravascular space for the assessment of autonomic compensation dynamics in the peripheral circulation. The device has a finger probe with a laser and optical detector that can provide single wavelength illumination capture remitted light to provide a temporally and spatially dense record of physiological information such as fluid volume in the capillaries.

### BACKGROUND OF THE INVENTION

### 1. Field of the Invention

The present invention relates to physiological measurement devices and, more particularly, to a device that can non-invasively measure the fluid content of intravascular spaces and autonomic compensation dynamics in peripheral circulation.

### 2. Description of the Related Art

Blood pressure, an essential vital sign, exists because the heart muscle squeezes the blood into the arteries. Autonomic compensation refers to unconscious and involuntary physiological responses to external and internal stresses and stimulation. These responses include redirection of blood flow to affect thermoregulation, systemic perfusion, oxygenation of all tissues, and to maintain all aspects of cardiovascular homeostasis generally. The early detection and characterization of vital signs associated with autonomic compensation can improve the diagnosis and treatment of various indications, e.g., hemorrhage and internal bleeding when there is no external injury, a leading cause of death world-wide.

### BRIEF SUMMARY OF THE INVENTION

The present invention can provide earlier detection and characterization of vital signs associated with autonomic compensation for the purposes of improving diagnosis and treatment of conditions such as hemorrhage and internal bleeding. A device according to the present invention can perform conditional probability analysis involving various sets of physiological vital sign data streams to characterize, display, and thereby predict in real time, the dynamics of autonomic compensation in different medical venues, e.g., dialysis, bleeding, and exercise/performance. Vital sign data can also be archived and referenced to a single time-base in a manner that allows for digital searching and studies that can improve health care in all settings.

More specifically, in a first embodiment, the invention may be a non-invasive device for measuring vital signs that has a finger probe, a laser source coupled to the finger probe to emit a predetermined amount of light at a predetermined wavelength from the finger probe, and an optical detector coupled to the finger probe and configured to detect single elastically scattered light and single inelastically scattered light from a finger of a patient positioned in the finger probe and output a signal representing an amount of elastically scattered light and an amount of inelastically scattered light received by the optical detector. A processing module is operatively coupled to the laser source and the optical detector to control the laser source and to receive the signal representing an amount of elastically scattered light and an amount of inelastically scattered light received by the optical detector, wherein the processing module is programmed to calculate a vascular volume for the finger positioned in the probe over a predetermined period of time and determine an amount of fluid removed from the patient over the predetermined period of time based on the calculation of the vascular volume over time. The optical detector may be configured to detect any fluorescence and any Raman spectra emitted from the finger of the patient. The device may further comprise a wireless communication interface coupled to the processing module. The device may further comprise a power supply coupled to the processing module, the optical detector, the finger probe, and the laser. The predetermined wavelength may be 830 nanometers. The processing module may be programmed to operate a laser driver of the laser source. The processing module may be programmed to include a timing circuit controlling timing of operation of the laser source and timing of the receipt of elastically scattered light and inelastically scattered light by the optical detector. The processing module may be programmed to be calibrated using fluid removal values obtained during dialysis of the patient.

In another embodiment, the invention may be a method of non-invasively measuring vital signals that begins with positioning a finger of a patient in a finger probe having a laser source coupled to the finger probe and an optical detector coupled to the finger probe. The laser source is operated to emit a predetermined amount of light at a predetermined wavelength from the finger probe. Any elastically scattered light and any inelastically scattered light from the finger of the patient is detected with the optical detector. The optical detector then outputs a signal that represents an amount of elastically scattered light and an amount of inelastically scattered light. The signal is then processed with a processing module to calculate a vascular volume for the finger positioned in the probe over a predetermined period of time and determine an amount of fluid removed from the patient over the predetermined period of time based on the calculation of the vascular volume over time.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a block diagram of the components of a device according to the present invention.

FIG. 2 is a diagram of the components of a processing module for use with a device according to the present invention.

FIG. 3 is a graph of patient data that can be determined by a device according to the present invention.

FIG. 4 is a graph of measured fluid loss versus reference fluid removed that can be determined by a device according to the present invention.

FIG. 5 is an example of a patient display that can be supported by the device according to the present invention.

FIG. 6 is a chart of vital signs being monitored by a device according to the present invention that provides additional patient information.

FIG. 7 is an example of a patient display that can be supported by a mobile device according to the present invention.

FIG. 8 is a flowchart of a process of monitoring a patient using a device according to the present invention.

### DETAILED DESCRIPTION OF THE INVENTION

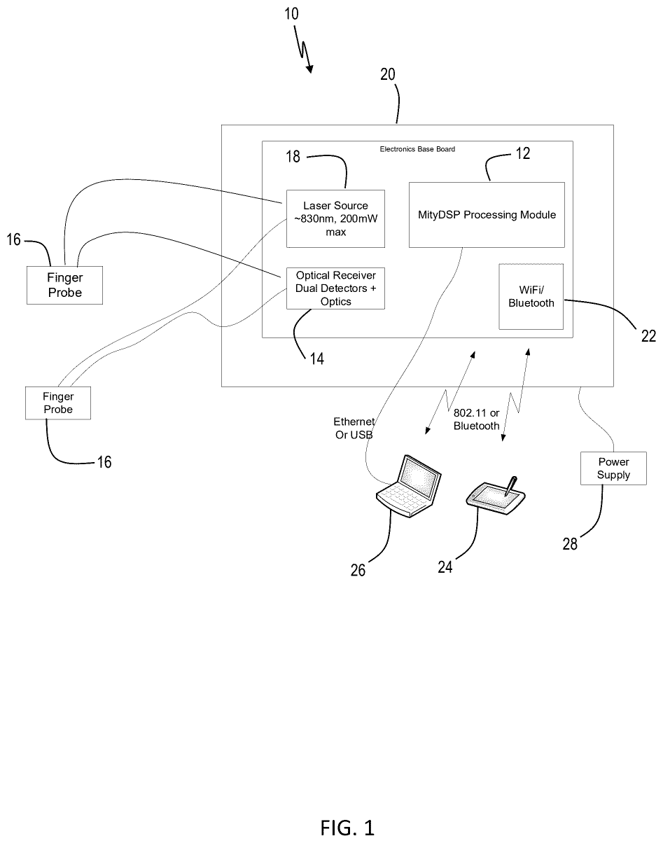

Referring to the figures, wherein like numerals refer to like parts throughout, there is seen in FIG. 1 a device 10 according to the present invention. Device 10 comprises a processing module 12 coupled to an optical detector 14 that can collect data received from one or more finger probes 16. A laser source 18 is used to provide single wavelength light (such as 830 nm) at a predetermined power level (such as 200 mW max) into a finger inserted into each probe 16. Processing module 12, optical detector 14, and laser source 18 may be provided on a single electronics board 20 along with a wireless communication interface 22 to allow wired and wireless remote connectivity to one or more external computing devices 24 such as a smartphone. Wired communication to a host computer 26, such as Ethernet, may be provided. Device 10 may thus be interconnected to other physiological monitors and devices for integration of sensor data from multiple sources (such as electrocardiograms, smart fitness wearable devices, pulse oximeters, etc.) into a single display for easy viewing, for programmed interpretation of combined data, and for other applications such as fitness monitoring and medical diagnostics. Device 10 additionally includes a power supply 28 for providing power to processing module 12, optical detector 14, and laser source 18.

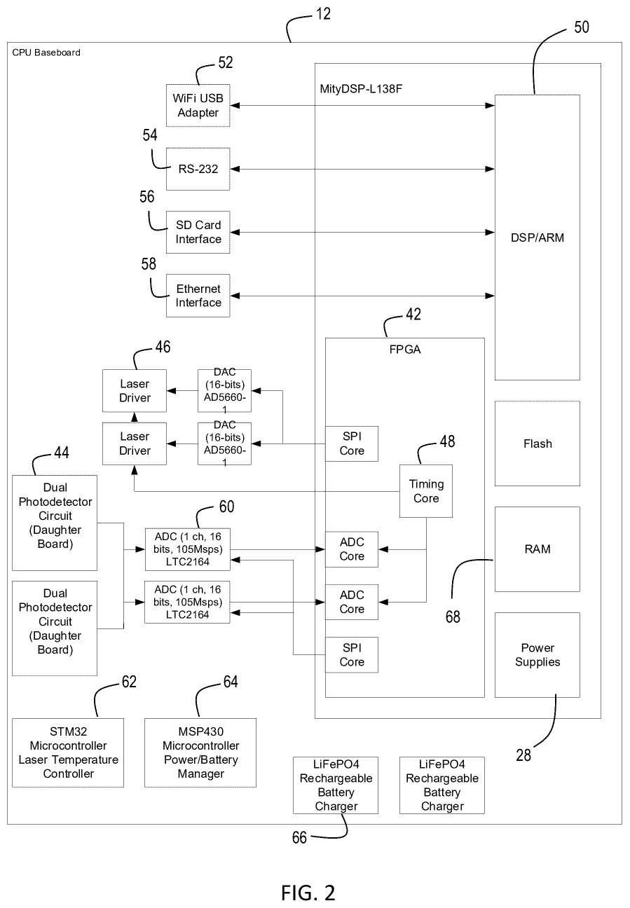

Referring to FIG. 2, processing module 12 includes an FPGA 42 for managing the photodetector circuits 44 associated with finger probes 16, the laser drivers 46 associated with laser source 18 and includes a timing circuit 48 controlling the delivery of laser illumination and detection. Processing module further includes a digital signal processor 50 as well as interfaces for connecting to one or more of a WiFi adaptor 52, an RS-232 port 54, a memory card 56, and an ethernet interface 58. As is known in the art, FPGA 42 may include analog to digital converter circuits 60 for receiving analog signals from photodetectors and converting into digital signals for electronic processing. In addition, processing module 12 may include a laser temperature controller 62 and a power manager 64 as well as rechargeable battery circuits 66 and standard memory modules 68.

Finger probes 16 are configured to be placed on a human finger and secured in place, such as by adjustable Velcro straps, to produce the most detectable pulse as reported by the patient or use of a finger-clip that can self-adjust pressure based on e.g., acoustic feedback. This is known from oscillometry to occur at the “mean arterial pressure” or MAP for that patient at that time. At this pressure loosen the Velcro very slightly to barely retain the perception of the pulse (by the patient) and to avoid occluding or otherwise impeding the flow of blood through the region covered i.e., probed by the light. Having placed and secured the probe(s), it is essential to avoid movement of the probe(s) relative to the skin surface to avoid measurement artifacts.

With the probe(s) in place, e.g., the two probes illustrated, each probe must be supplied with light from a suitable source (laser as shown or light emitting diode (LED) and then the remitted light must be collected and returned to an optical receiver. At a minimum, (SansR embodiment of the present invention) the optical receiver separates the remitted light into two signals i.e., elastically scattered light (EE) and inelastically scattered light (IE). The EE and IE optical signals that are then converted to the analog electronic domain using two suitable photodetectors in addition to the other optical components. These analog signals are transferred to the processing module for digitization, storage, and algorithm execution. Although connections are not shown explicitly the processing module also executes a variety of functions like wireless and ethernet I/O, temperature maintenance for the laser(s), external power/battery supply and similar tasks. If the optical receiver module contains dispersive optical element(s) and multichannel light detection, then a device capable of fluorescence and/or Raman spectroscopy (AvecR embodiment of the present invention) becomes possible and much more detailed information can be gleaned from the remitted light besides fluid volume. In either the SansR or AvecR embodiments of the present invention, autonomic compensation, and pulse rate (PR) information are available through the time dependence of the fluid volume (VV) measurements. PR requires the laser to operate at a much larger duty cycle than for pure VV measurements alone and battery size/presence must be considered if those two vital signs i.e., PR and VV are to be provided simultaneously by the same device.

The information gleaned by finger probes 16 are processed by processing module 12 to determine the desired physiological characteristics. For example, the volume fractions for RBCs, plasma and surrounding static tissues sum to unity in formula [1], implying that there are no voids even when there is applied pressure or temperature changes, as indicated by [2], that deform static tissue and cause movement of mobile tissues i.e., blood. This is summarized in equations [1] and [2] using ϕ for each of the volume fractions, i.e., RBCs, plasma, and static tissue in the probed volume.

$$1 = {ϕ}_{r} + {ϕ}_{p} + {ϕ}_{s} \qquad [ 1 ]$$

$$0 = d {ϕ}_{r} + d {ϕ}_{p} + d {ϕ}_{s} \qquad [ 2 ]$$

On a most coarse level, the composition of any blood sample can be defined in terms of the volumes of two components, plasma content and RBC content. Hematocrit (Hct) is defined as the percentage of blood by volume that is RBCs as indicated by [3].

$$Hct = {ϕ}_{r} / ( {ϕ}_{r} + {ϕ}_{ρ} ) \qquad [ 3 ]$$

For clinical reasons, only the plasma content is sought by device 10, an extensive property which is also termed vascular volume (VV). Unless a person is anemic at the systemic level, the absolute value of the Hct or plasma content can vary significantly without producing actionable physiological indication. An absolute scale for VV is necessary but autonomic compensation occurs within a range of normal homeostatic vital signs for each person. VV is defined by measurements on the patient immediately before monitoring begins using the sum of d, e, and f in equation [4] i.e., the ratios in [4] are both equal to 1 since EE=EE₀ and IE=IE₀. Therefore, at time equal 0:

$${ϕ}_{p} ( 0 ) = d + e + f \qquad [ 4 ]$$

The volume probed by the light is assumed to be constant and so the calculated volume (extensive) of plasma is always proportional to ϕₚ. VV can then be defined as 100×(ϕₚ(t)/ϕₚ(0)) that is, as a percentage of the initial value of ϕₚ. This issue is also addressed below when it is shown how naturally the parameters can be obtained in a dialysis or by extension, other medical venues.

Hct itself is unitless and an intensive quantity, being defined by the ratio of the plasma content to the red blood cell content, both defined in terms of volume, an extensive property. Calibration of the device is on e.g., independent measurements of Hct is 1) indirect and 2) requires extra work compared to what will be taught in this patent. It is indirect because Hct is not necessarily determined by the total intravascular volume and more laborious because to calculate Hct one must first obtain both ϕᵣ and ϕₚ. As devices such as CritLine which produces a continuous Hct data stream using analysis of extracorporeal blood during dialysis are no longer easily available, there is a continuing need to improve technology for calibrating devices for noninvasive in vivo blood and tissue analysis.

$$EE = {ϑ}_{1} + {ϑ}_{2} {ϕ}_{p} + {ϑ}_{3} {ϕ}_{r} \qquad [ 5 ]$$

$$EE = {ϑ}_{1} + {ϑ}_{2} {ϕ}_{p} + {ϑ}_{3} {ϕ}_{r} \qquad [ 6 ]$$

$${φ}_{p} = d + e ( \frac{E E}{E {E}_{0}} ) + f ( \frac{I E}{I {E}_{0}} ) \qquad [ 7 ]$$

The fluid volume is determined by the state of dilation/constriction of the capillaries and the blood pressure i.e., the mean arterial pressure (MAP) and these states are mostly under autonomic control.

One clinical application of this invention is to obtain a temporally and spatially dense record of this fluid volume i.e., VV in the capillaries to allow observation, analysis and ultimately prediction of autonomic compensation. An advantage of the present invention is the preferred use of independent measurements of an extensive physical property to calibrate a device that measures an extensive physiological property. Unlike CritLine, fluid removed measurements are always available during dialysis for calibration and are directly connected to VV. Hct depends on both the amount of blood inside the tubes which determines the blood pressure, and their internal diameter which affects the blood viscosity in a manner that physically reduces/minimizes resistance to flow. While CritLine has demonstrated that improved medical outcomes come from tighter control, FRD-PVOH obtains more detailed and pertinent information because it works in vivo and so we expect even better outcomes.

Currently absolute MAP can only be measured continuously using the expensive, painful, and invasive arterial puncture that can also become infected like with all invasive procedures. VV measurements have been shown to reflect changes in MAP in various laboratory experiment contexts and we expect that in many cases this invention can be substituted for arterial puncture. VV and MAP when combined with pulse rate (PR) measurements, also enters into a display mode described below for specialized I/O in real-time situations e.g., during dialysis, search for blood loss, exercise and more.

The validity of equation [7] was demonstrated with radiation transfer theory and validated experimentally in a controlled in vitro setting.

$${φ}_{p} = d + e ( \frac{E E}{E {E}_{0}} ) + f ( \frac{I E}{I {E}_{0}} ) \qquad [ 7 ]$$

Note that EE₀ and IE₀ (collectively called “the zeros”) are measurements of EE and IE at time 0 that is, before monitoring begins, thereby partially calibrating the equation i.e., providing normalization to the individual patient, independent of the values of d, e, and f. Calibrating with respect to d, e, and f presents many options including which independent measurements are employed for reference and how these measurements are obtained. Fluid removed (FR) measurements during dialysis as described below correlate directly to the fluid component in the intravascular space because ideally no RBCs are removed during dialysis.

However, the same approach could be employed using whole blood draws as occurs during blood donation or transfusion. The location(s) of 1) noninvasive optical monitoring and 2) physical sampling (for reference) are important choices, resulting in empirical sensitivity to physiological processes that can be interpreted in terms of turbidity and fluid content and when AvecR is possible, more detailed hemical composition.

Although the absolute value of VV is important, much clinical value resides in the time variation of the VV, as a percentage of VV at the time=0 value manifesting the present state of homeostasis, defined by the amount of autonomic activity, as discerned by fluid removed determination (FRD). Thus, for that purpose we choose to illustrate a particular FRD calibration using the data and approach illustrated in Table 1.

TABLE 1
Data for FRD calibration i.e., calculation
of parameters d, e, f from equation [1]

| FR | Vₚ assume | | | | | | |
|---|---|---|---|---|---|---|---|
| (mL) | 4 L total | V_{RBC} | Vₜₒₜₐₗ | ϕ_{RBC} | ϕₚ | EEₐᵥₑ | IEₐᵥₑ |
| 20 | 2.67 | 1.33 | 4 | 0.3325 | 0.6675 | | |
| 70 | 2.62 | 1.33 | 3.95 | 0.33671 | 0.66329 | | |
| 110 | 2.58 | 1.33 | 3.91 | 0.34015 | 0.65985 | 39962.9 | 7373.5 |
| 160 | 2.53 | 1.33 | 3.86 | 0.34456 | 0.65544 | 39634.5 | 7207.1 |
| 220 | 2.47 | 1.33 | 3.8 | 0.35 | 0.65 | 41207.0 | 6720.8 |
| 270 | 2.42 | 1.33 | 3.75 | 0.35467 | 0.64533 | 37514.9 | 6194.9 |
| 310 | 2.38 | 1.33 | 3.71 | 0.35849 | 0.64151 | 37771.4 | 6425.5 |
| 360 | 2.33 | 1.33 | 3.66 | 0.36339 | 0.63661 | 38697.9 | 6091.2 |

To determine the values of the empirical parameters d, e, and f, we require at least three reference values of ϕₚ and the corresponding i.e., values of EE and IE. Given that the patient is undergoing dialysis, ϕₚ at any time can be obtained using 1) the assumption that the total blood content of the patient is 4.0 liters and 2) the amount of intravascular fluid present at each time point is 4.0−FR where is FR is the value of fluid removed at that time as reported by the dialysis machine. The volume fraction ϕₚ at each time point is given by (4−FR)/4. If FR is known for at least 3 different times during the dialysis procedure, along with the corresponding EE and IE values at those time points, and of course EE₀ and IE₀, then ϕₚ can be calculated and the parameters d, e, and f can be obtained by bilinear regression of the normalized EE and IE (normalized to EE₀ and IE₀ as indicated by [1]) onto the values of ϕₚ.

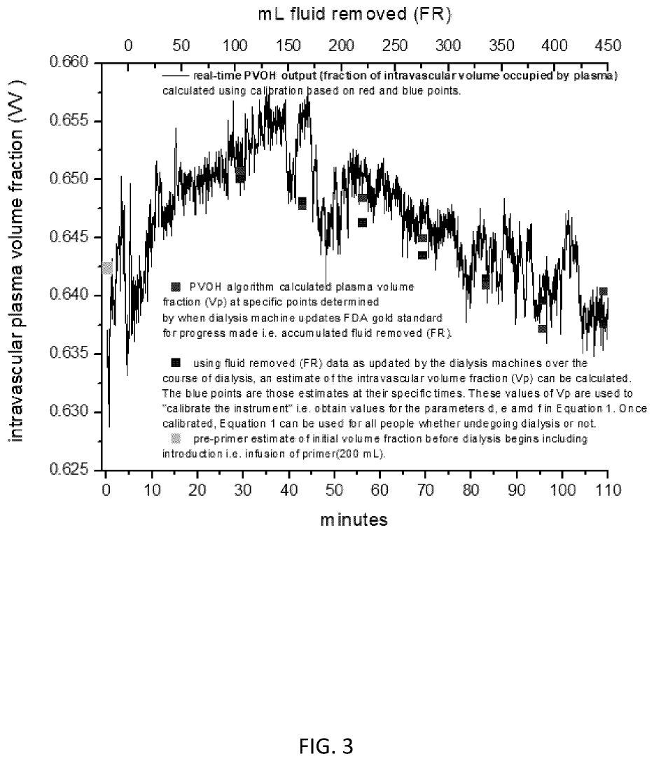

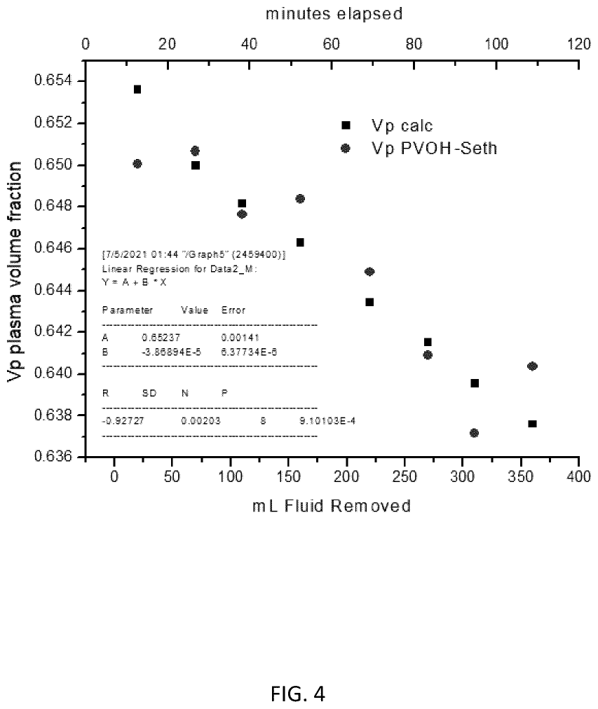

FIG. 3 shows the device output and calibration points used for data being obtained from a person with low body mass index during hemodialysis. The values of d, e, and f obtained by regression can then be used for other patients going forward. Or, if a person is monitored regularly during dialysis, or some other procedure that could give fluid volumes analogous to FR, then a set of d, e, and f parameters can be optimized for that specific person. An AI or machine learning approach could combine the longitudinal/transverse variation of these parameters, involving multiple parallel physiological/physicochemical data streams to synthesize a unified and more complete representation of the patient's condition. FIG. 4 shows the PVOH/FRD Measured Fluid Loss vs. Reference Fluid Removed.

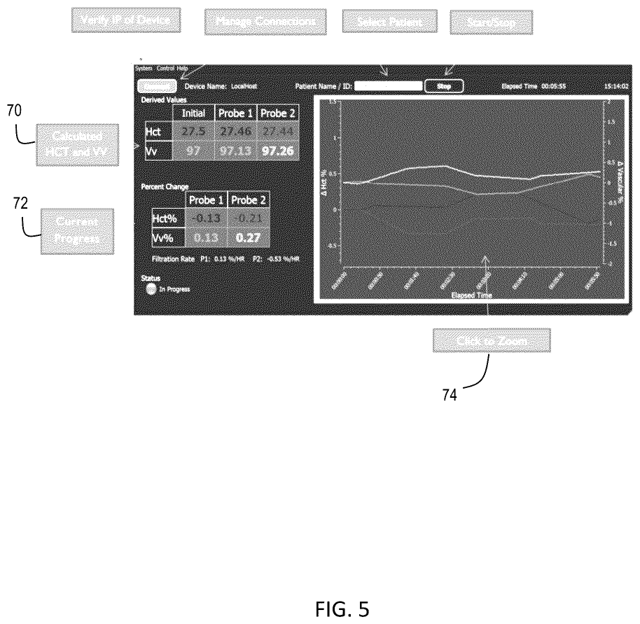

Referring to FIG. 5, calculations provided by device 10 may be provided in real-time to medical personnel for use in diagnosis and treatment. For example, host computer 26 or wireless connected device 24 may be programmed to display the real time calculations provided by device 10. As seen in FIG. 5, the calculated hematocrit 70 and vascular volume 72 may be displayed as calculated values or in an interactive graph 74. Standard security features such as device recognition, connection management, patient selection, and remote commanding of device 10 may also be provided.

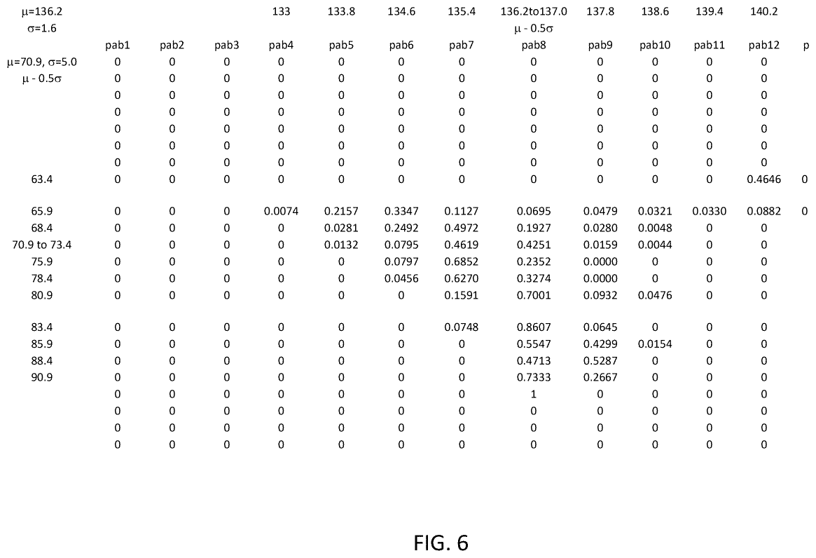

Referring to FIG. 6, data can also be provided in a chart that allows for a determination of sustainable exertion or load, such a grid involving at least two vital signs being monitored. FIG. 6 is one specific example of a general approach for creating this kind of grid. Although based on actual Vₚ and PR measurements on a test subject undergoing a gentle thermoregulation challenge, other combinations of e.g., vital signs and deviations from homeostasis would yield a view of physiology projected onto different essential functions i.e., perfusion and oxygenation of all tissues. Given that at least 5 different vital signs exist for humans, it is not possible to represent measurements of them all simultaneously in a 2-dimensional view. But they are all interrelated, so there are constraints between the possible measured values of such quantities for normal homeostasis. This is the underlying basis for using the conditional probability matrix (CPM) as the grid, as was calculated using simultaneous PR and Vₚ temporal (10 Hz i.e., both 0.1 second data interval) streams in FIG. 6.

It is possible to calculate the conditional probability matrix (CPM) of these two data streams (e.g., Vₚ and PR as in FIG. 6) as well as their respective means (m) and standard deviations (s). To initialize the display this will be done as a monitoring session begins e.g., for perhaps 5 minutes or 3000 data point pairs whichever comes first, or in case of emergency, via manual override using whatever data has already been collected, and is updated usually in e.g., 15 second intervals or faster because the data streams are/can be temporally dense (acquisition bandwidth up to 50 Hz) relative to many normal physiological timescales. These initial m and s, or updated versions as data collection proceeds, will be used to implement two of the three possible display modes for the CPM claimed by this patent. The third mode simply involves absolute VV and PR values.

The CPM can be explained by reference to FIG. 6. The first non-zero matrix element or “bin” in the upper left corner of FIG. 6, in labelled column “pab4”, has the value 0.0074 and that value corresponds to the probability that the Vₚ has the value 133<Vp<133.8% of initial value when monitoring session began, on the condition that 65.9<PR<68.4 beats/minute. The entries in this CPM are calculated by direct sorting of the paired input data streams into the bins such that each time a specific data pair occurs, the corresponding bin is incremented by 1 so that when all the data is included, the bin contains the number of occurrences of that combination of Vp and PR. Proper normalization is required to obtain probabilities, with each row in FIG. 6 being normalized by summing occurrences in all bins spanning all observed values of Vₚ but unconditionally for PR, i.e., dividing all entries in that row by the total number of occurrences of that PR. The CPM is updated by sorting new data into either 1) the same bins (i.e., widths and centers) created at initialization or 2) using bins based on updated PR and Vp m and s values as new data is collected.

In the example shown, the bin widths and centers are defined by the existing m and s values, perhaps from initialization, and so the display is automatically scaled to the autonomic performance of each patient, and the particular mode of deviation from homeostasis i.e., thermal, orthostatic or other means. In FIG. 6, the widths are in units of 0.5 s or half of a standard deviation with the center bin chosen to be centered on the unconditional m for that variable. of the unconditional average of either. This is true regardless of whether the means and standard deviations used are initialization or updated versions. This could be an option selectable by medical personnel depending on what point in their observations they choose as a point of reference.

Using updated or not updated m and s as two different display modes, then the third mode is “absolute” where the same bin centers and widths are used but labelled in absolute units. This could be used to make transverse comparison of response to stimuli across patients. In an exercise science context, the performance of an elite athlete could be compared longitudinally or transversely to assess training efficacy and so the display has button select ability to switch between absolute and the statistically scaled display labelling modes easily.

Since data streams are continuously accumulating, an icon or other easily seen identifier may be used in the real-time display, overlaying the location in the CPM based on the most recent read of the vital signs. A monitoring session would be archivable as a trajectory through a space defined by the available vital signs. All CPM combinations involving vital signs, i.e., SpO₂ and PR, or Vp and SpO₂, can be easily calculated in analogy to the example given in this application, and they could all be displayed simultaneously in a large visual display. However, certain combinations reflect deterministic connections between the quantities associated with the vital signs and these CPMs will have a specific form.

First, note that if the heart beats more often or more forcefully, then it is expected that the volume of blood, i.e., the Vₚ downstream, will increase. Moreover, the pressure required to cause blood movement increases with the average diameter of the blood vessels if the force being delivered by the heart is constant. Equating the Vₚ with the MAP, as has been observed empirically, and the product of PR and the cardiac ejection volume with a net flow rate, Poiseuille's equation indicates Vp and PR are linearly related. FIG. 6 shows exactly that behavior with actual data and the following formula demonstrates why this is the case.

To calculate the (thermodynamic) work done by the heart and vasculature to pump blood through the entire body begin with Poiseuille's equation for the flow of liquids in tubes.

$$p = \frac{fl 8 η}{π {r}^{4}}$$

f=rate of blood flow

$$( \frac{cc}{sec} )$$

in vessel with length l (for capillaries 0.05 cm), internal radius of r (cm), with viscosity η(≈2.8 cp) and pressure drop across length of vessel of p (dynes/cm²)

$$f = {V}_{e} {R}_{p} \qquad [ 8 ]$$

Vₑ is left ventricular ejection volume (cc) and Rₚ is pulse rate (pulses/sec)

$$Work ( erg / sec ) = pf \qquad [ 9 ]$$

$$pf = \frac{{f}^{2} l8 η}{π {r}^{4}} = 1.45 E 7 erg / sec = 30 Cal / day$$

Empirical results from PVOH/FRD device indicate that in homeostatic resting state:

$$MAP ∝ VV$$

Therefore, associating MAP with p in equation [9] and f with Vₑ and Rₚ as in [8]:

$$\begin{matrix}{MAPV}_{e} {R}_{p} = \frac{{V}_{e}^{2} {R}_{p}^{2} l 8 η}{π {r}^{4}} \\ VV / {R}_{p} = \frac{{V}_{e} l 8 η}{π {r}^{4}}\end{matrix} \qquad [ 10 ]$$

Now, VV/Rp is the ratio of the VV from PVOH/FRD and Rp is the pulse rate. And to the extent the physiology is running homeostatic, the right-hand side of [10] is constant.

Thus, the slope of the CPM boundary at low VV range is also a constant, as observed.

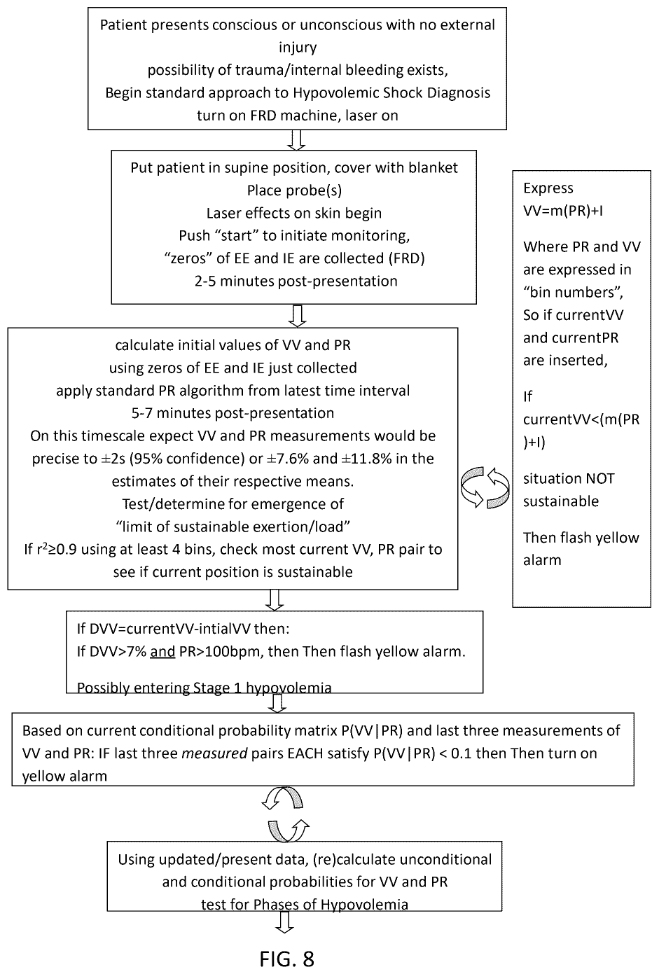

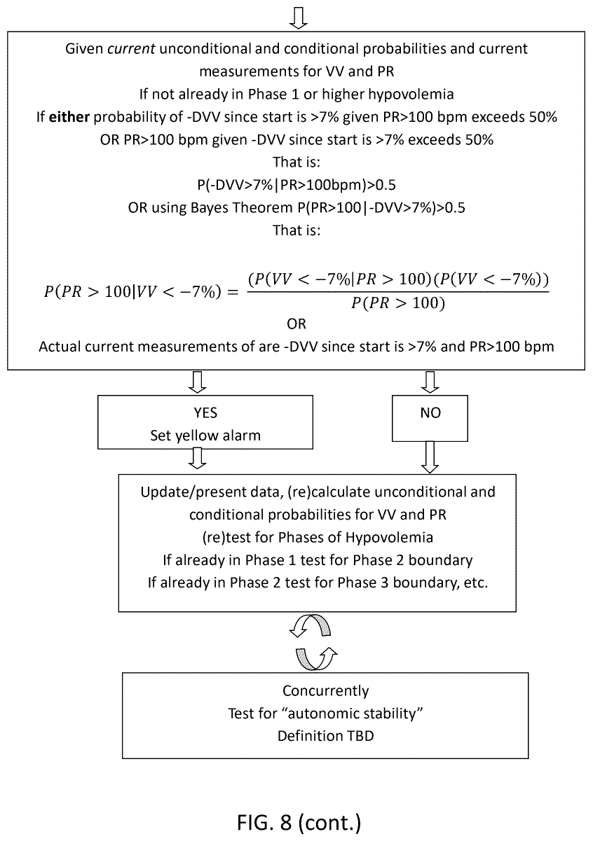

Connecting the bins with contents 0.0074, 0.0132, 0.456, 0.0748, 0.733, it is possible to perceive a “boundary” between sustainable cardiovascular physiology (to the right) and the unsustainable condition (to the left) where the Vp cannot increase with increasing PR or ejection volume. FIG. 8 shows a flow chart that would be appropriate for using FRD in the diagnosis of internal bleeding. Note that using Bayes Theorem to identify situations which could progress to an event requires the CPM but also the univariate unconditional distributions.

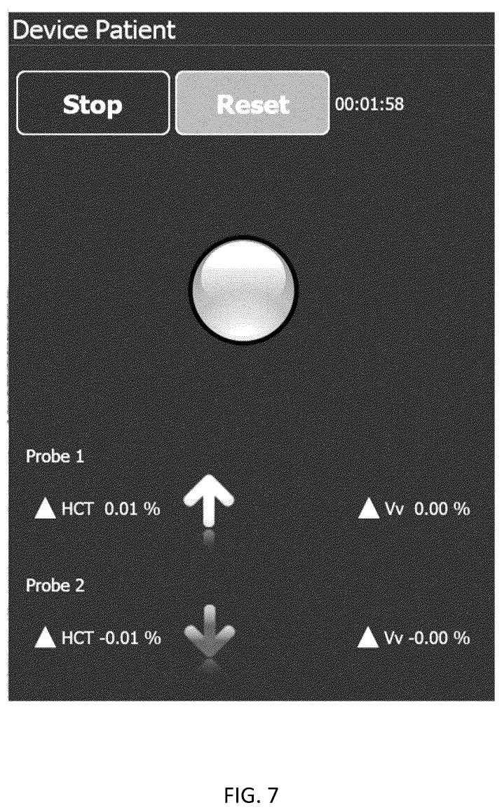

Finally, a mobile application having a display such as that seen in FIG. 7 may be used for critical care trauma scenarios for a quick assessment of potential hemorrhage using hematocrit and vascular volume information provided by device 10. While the dialysis embodiment provides expansive detail for all aspects of the data collection and presentation, the trauma embodiment and the protocol of the present invention seen in FIG. 8 is intentionally more streamlined in its presentation to focus on device status, hematocrit trend and monitoring/notification of battery supply as it is intended to be used as a mobile unit for easy assessment of blood loss.

What is claimed is:

1. A non-invasive device for measuring vital signs, comprising:
a finger probe;
a laser source coupled to the finger probe to emit a predetermined amount of light at a predetermined wavelength from the finger probe;
an optical detector coupled to the finger probe and configured to detect any elastically scattered light and an inelastically scattered light from a finger of a patient positioned in the finger probe and output a signal representing an amount of elastically scattered light and an amount of inelastically scattered light received by the optical detector; and
a processing module operatively coupled to the laser source and the optical detector to control the laser source and to receive the signal representing an amount of elastically scattered light and an amount of inelastically scattered light received by the optical detector, wherein the processing module is programmed to calculate a vascular volume for the finger positioned in the finger probe over a predetermined period of time and determine an amount of fluid removed from the patient over the predetermined period of time based on the calculation of the vascular volume over time.

2. The non-invasive device of claim 1, wherein the optical detector is configured to detect any fluorescence and any Raman spectra emitted from the finger of the patient.

3. The non-invasive device of claim 1, further comprising a wireless communication interface coupled to the processing module.

4. The non-invasive device of claim 1, further comprising a power supply coupled to the processing module, the optical detector, the finger probe, and the laser source.

5. The non-invasive device of claim 1, wherein the processing module is further programmed to calculate pulse rate.

6. The non-invasive device of claim 5, further comprising a display in communication with the processing module, wherein the processing module is programmed to cause the display to show the amount of fluid removed from the patient over time in combination with the pulse rate.

7. The non-invasive device of claim 1, wherein the processing module is programmed to include a timing circuit controlling timing of operation of the laser source and timing of the receipt of elastically scattered light and inelastically scattered light by the optical detector.

8. The non-invasive device of claim 1, wherein the processing module is programmed to be calibrated using fluid removal values obtained during dialysis of the patient.

9. A method of non-invasively measuring vital signals, comprising the steps of:
positioning a finger of a patient in a finger probe having a laser source coupled to the finger probe and an optical detector coupled to the finger probe;
operating the laser source to emit a predetermined amount of light at a predetermined wavelength from the finger probe;
detecting any elastically scattered light and an inelastically scattered light from the finger of the patient with the optical detector;
outputting a signal from the optical detector that represents an amount of elastically scattered light and an amount of inelastically scattered light received by the optical detector, and
processing the signal from the optical detector with a processing module to calculate a vascular volume for the finger positioned in the finger probe over a predetermined period of time and determine an amount of fluid removed from the patient over the predetermined period of time based on the calculation of the vascular volume over time.

10. The method of claim 9, wherein the optical detector further detects any fluorescence and any Raman spectra emitted from the finger of the patient.

11. The method of claim 9, further comprising the step of using a wireless communication interface coupled to the processing module to transmit the calculation of vascular volume to a remote device.

12. The method of claim 9, wherein the finger probe further comprises a power supply coupled to the processing module, the optical detector, and the laser source.

13. The method of claim 9, further comprising the step of displaying the amount of fluid removed from the patient over time on a display in communication with the processing module.

14. The method of claim 13, further comprising the step of determining a pulse rat of the patient.

15. The method of claim 14, further comprising the step of displaying the amount of fluid removed from the patient over time in combination with the pulse rate.
